(19) United States
(12) Patent Application Publication
(10) Pub. No.: US 2012/0204876 A1
(43) Pub. Date: Aug. 16, 2012

(54) FLUID FLOW CONTROL APPARATUS AND METHOD AND PATIENT INTERFACE DEVICE EMPLOYING SAME

(75) Inventor: JEROME MATULA, Jr., APOLLO, PA (US)

(73) Assignee: KONINKLIJKE PHILIPS ELECTRONICS N.V., EINDHOVEN (NL)

(21) Appl. No.: 13/503,448

(22) PCT Filed: Sep. 28, 2010
(86) PCT No.: PCT/IB10/54367

§ 371 Date: Apr. 23, 2012

Related U.S. Application Data
(60) Provisional application No. 61/255,234, filed on Oct. 27, 2009.

(51) Int. Cl. A61M 16/06; F17D 3/00

(52) U.S. Cl. 128/205.25; 137/1

(57) ABSTRACT

A fluid flow control apparatus for use in, for example, a patient interface, is provided that includes a main conduit structured to receive a pressurized fluid and a nozzle assembly coupled to the main conduit. The nozzle assembly includes an external nozzle element having a contoured top surface and a main bore in fluid communication with an interior of the main conduit, an internal nozzle element received within the main bore, wherein a gap is provided between the internal nozzle element and the external nozzle element, and a switching element selectively moveable between a first position and a second position, wherein in the first position, the switching element is positioned below the contoured top surface of the external nozzle element, and wherein in the second position, at least a portion of the switching element is positioned adjacent to or above the contoured top surface of the external nozzle element.

### CROSS-REFERENCE TO RELATED APPLICATIONS

This patent application claims the priority benefit under 35 U.S.C. §371 of international patent application no. PCT/IB2010/054367, filed Sep. 28, 2010, claims the priority benefit under 35 U.S.C. §119(e) of U.S. Provisional Application No. 61/255,234 filed on Oct. 27, 2009, the contents of which are herein incorporated by reference.

### BACKGROUND OF THE INVENTION

1. Field of the Invention

The present invention relates to the control of the flow of fluids, such as liquids or gases, and, more particularly, to an apparatus for selectively controlling the direction of flow of a fluid that may be employed in, for example, a respiratory patient interface device.

2. Description of the Related Art

Numerous devices for transporting, distributing and delivering pressurized fluids, such as gases and liquids, are known in the art. Such devices have many applications, including, without limitation, industrial, scientific and medial applications. While many known devices include a valve, exhaust assembly or similar structure for controlling when and how the pressured fluid is allowed to be released from the device, it would be desirable in some applications to be able to selectively control and possibly reverse the direction of the flow of the fluid in and around the device. Current devices do not provide such a capability.

For example, there are numerous situations where it is necessary or desirable to deliver a flow of breathing gas non-invasively to the airway of a patient. Such non-invasive therapies ventilating a patient using a technique known as non-invasive ventilation, and delivering a continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure. Non-invasive ventilation and pressure support therapies involve the placement of a respiratory patient interface device including a mask component, which is typically a nasal mask that covers the nose, a nasal cushion having nasal prongs that are received within the patient's nares, a nasal/oral mask that covers the nose and mouth, or full face mask that covers the patient's face, on the face of a patient. The respiratory patient interface device interfaces the ventilator or pressure support device with the airway of the patient, so that a flow of breathing gas can be delivered from the pressure/flow generating device to the airway of the patient. It is known to provide such patient interface device with an exhaust port to allow the patient's expired gas to be vented to atmosphere. Current patient interface devices do not include exhaust ports that allow the direction of fluid flow around the port to be selectively controlled.

### SUMMARY OF THE INVENTION

In one embodiment, a patient interface device is provided that includes: (a) a cushion, (b) a mask frame supporting the cushion, (c) a patient circuit coupled to the mask frame adapted to carry a flow of gas, and (d) a fluid flow control apparatus coupled to the mask frame or the patient circuit. The fluid flow control apparatus includes a main conduit structured to receive a pressurized fluid and a nozzle assembly coupled to the main conduit. The nozzle assembly includes an external nozzle element having a contoured top surface and a main bore in fluid communication with an interior of the main conduit, an internal nozzle element received within the main bore, wherein a gap is provided between the internal nozzle element and the external nozzle element, and a switching element selectively moveable between a first position and a second position, wherein in the first position, the switching element is positioned below the contoured top surface of the external nozzle element, and wherein in the second position, at least a portion of the switching element is positioned adjacent to or above the contoured top surface of the external nozzle element. In one particular embodiment, the at least a portion of the switching element is positioned above the contoured top surface of the external nozzle element.

The external nozzle element may have an annular shape and the switching element may be an annular body. The contoured top surface may be semicircular and may extends along only half of the entire top surface of the external nozzle element.

Alternatively, the external nozzle element may have a rectangular shape, wherein the contoured top surface includes a first contoured surface portion on a first side of the external nozzle element and a second contoured surface portion on a second side of the external nozzle element opposite the first side. In another alternative, the external nozzle element may include a plurality of semi-cylindrical sections coupled to one another by a plurality of curved joining sections, wherein the contoured top surface includes a plurality of contoured surface portions, each contoured surface portion is provided on the top of a respective one of the semi-cylindrical sections.

The contoured top surface may include a convex portion adjacent to an inner wall portion of the external nozzle element, and a concave portion adjacent to the convex portion. In one particular embodiment, the internal nozzle element includes a conical top portion having a flat base connected to an external wall, the external wall extending downwardly from the flat base toward the main conduit, wherein the gap is between the external wall and the convex portion and the inner wall portion of the external nozzle element. The conical top portion may be coupled to a cylindrical bottom portion, wherein the cylindrical bottom portion is received and held within an internal wall of the external nozzle element. The position of the internal nozzle element relative to the external nozzle element may be selectively adjustable. For example, both the cylindrical bottom portion and the internal wall may be threaded for this purpose.

A method of controlling fluid flow is also provided that includes providing a fluid flow control apparatus having a main conduit and a nozzle assembly coupled to the main conduit, the nozzle assembly including an external nozzle element having a contoured top surface and a main bore in fluid communication with an interior of the main conduit and an internal nozzle element received within the main bore, wherein a gap is provided between the internal nozzle element and the external nozzle element. The method also includes steps of providing a pressurized gas within the main conduit in an initial state, wherein in the initial state: (i) an exhaust flow flows forward and out of the nozzle assembly in a direction away from a top center of the nozzle assembly, and (ii) ambient gas surrounding the nozzle assembly is drawn from a perimeter of the nozzle assembly and toward the top center of the nozzle assembly, causing the fluid flow control apparatus to move to a switched state by disrupting a flow of the ambient gas toward the top center of the nozzle assembly, wherein in the switched state: (i) an ambient gas intake flow is present and flows in a backward direction toward the top center of nozzle assembly, (ii) ambient gas surrounding the nozzle assembly flows away from the top center of nozzle assembly and toward the perimeter of the nozzle assembly, and a second exhaust flow flows out of the nozzle assembly and toward the perimeter of the nozzle assembly.

In another embodiment, a patient interface device is provided that includes: (a) a cushion, (b) a mask frame supporting the cushion, (c) a patient circuit coupled to the mask frame adapted to carry a flow of gas, and (d) a fluid flow control apparatus coupled to the mask frame or the patient circuit. The fluid flow control apparatus in this embodiment includes a main conduit structured to receive a pressurized fluid, and a nozzle assembly coupled to the main conduit. The nozzle assembly an external nozzle element having a contoured top surface and a main bore in fluid communication with an interior of the main conduit, wherein the contoured top surface comprises a convex portion adjacent to an inner wall portion of the external nozzle element, and a concave portion adjacent to the convex portion, and an internal nozzle element received within the main bore, wherein a gap is provided between the internal nozzle element and the external nozzle element.

The invention also provides a method of controlling fluid flow that includes a step of providing a fluid flow control apparatus having a main conduit and a nozzle assembly coupled to the main conduit, the nozzle assembly including an external nozzle element having a contoured top surface and a main bore in fluid communication with an interior of the main conduit and an internal nozzle element received within the main bore, wherein a gap is provided between the internal nozzle element and the external nozzle element. The method further includes providing a pressurized liquid within the main conduit in an initial state, wherein in the initial state: (i) the pressured liquid flows though the gap and out of the nozzle assembly, (ii) the nozzle assembly is positioned above a top surface of a body of liquid, (iii) a fluid flow in the body of liquid goes from outside a perimeter of the external nozzle element to inside the perimeter of the external nozzle element, and downward and away from the fluid flow control apparatus, and causing the fluid flow control apparatus to move to a switched state by introducing the nozzle assembly into the body of liquid, wherein in the switched state: (i) the pressured liquid flows though the gap and out of the nozzle assembly, (ii) the fluid flow in the body of liquid reverses and goes upward and toward the fluid flow control apparatus and from inside the perimeter of the external nozzle element to outside the perimeter of the external nozzle element. In one particular embodiment, the contoured top surface includes a convex portion adjacent to an inner wall portion of the external nozzle element, and a concave portion adjacent to the convex portion, and in the switched state, all of the convex portion and all or part of the concave portion of the contoured top surface lie beneath a top surface of the body of liquid.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of “a”, “an”, and “the” include plural referents unless the context clearly dictates otherwise.

### BRIEF DESCRIPTION OF THE DRAWINGS

FIGS. 1A and 1B show a system for providing a regimen of respiratory therapy to a patient according to one embodiment of the present invention;

FIG. 2 is a front elevational view of a fluid flow control apparatus according to one particular embodiment of the present invention;

FIG. 3 is a front cross-sectional view of the fluid flow control apparatus of FIG. 2;

FIG. 4 is an exploded front elevational view of the fluid flow control apparatus of FIG. 2;

FIG. 5 is an exploded front cross-sectional view of the fluid flow control apparatus of FIG. 2;

FIG. 6 is an exploded isometric view of the fluid flow control apparatus of FIG. 2;

FIGS. 7, 8 and 9 illustrate operation of fluid flow control apparatus 2 according to one embodiment;

FIGS. 10 and 11 illustrate operation of a slightly modified fluid flow control apparatus according to another embodiment;

FIG. 12 is an exploded view of a fluid flow control apparatus according to an alternative embodiment of the present invention;

FIG. 13 is an isometric view of an external nozzle element according to an alternative embodiment that may be used with an alternative fluid flow control apparatus;

FIG. 14A is a top plan view and FIG. 14B is a cross-sectional view of an alternative internal nozzle element for use with the external nozzle element of FIG. 13;

FIG. 15 is an isometric view of an external nozzle element according to another alternative embodiment that may be used with another alternative fluid flow control apparatus;

FIG. 16 is a top plan view of an alternative internal nozzle element for use with the external nozzle element of FIG. 15;

FIG. 17 is a top plan view of an alternative switching element for use with the external nozzle element of FIG. 15; and

FIG. 18 is a top plan view of a further alternative switching element for use with the external nozzle element of FIG. 15.

### DETAILED DESCRIPTION OF EXEMPLARY EMBODIMENTS

Directional phrases used herein, such as, for example and without limitation, top, bottom, left, right, upper, lower, front, back, and derivatives thereof, relate to the orientation of the elements shown in the drawings and are not limiting upon the claims unless expressly recited therein. As employed, herein, the statement that two or more parts or components are “coupled” together shall mean that the parts are joined or operate together either directly or through one or more intermediate parts or components. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

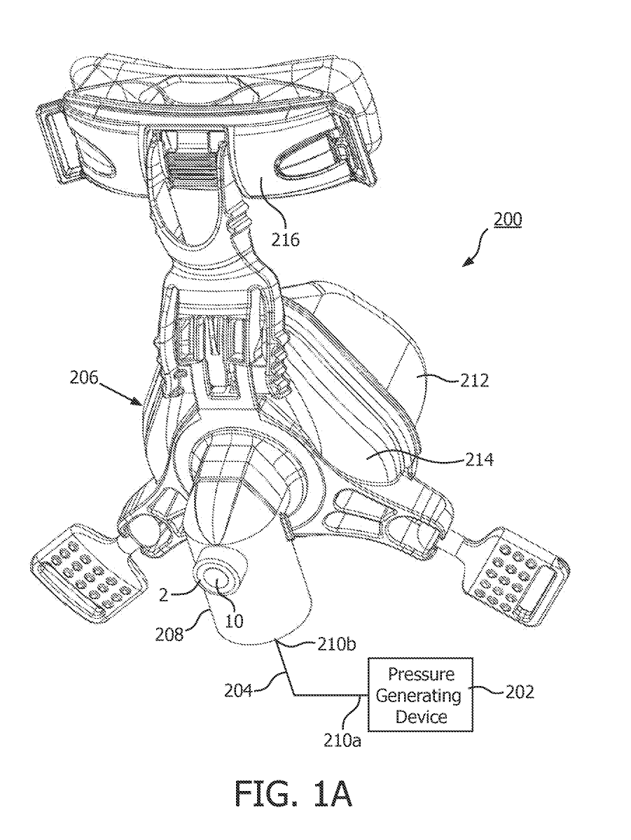

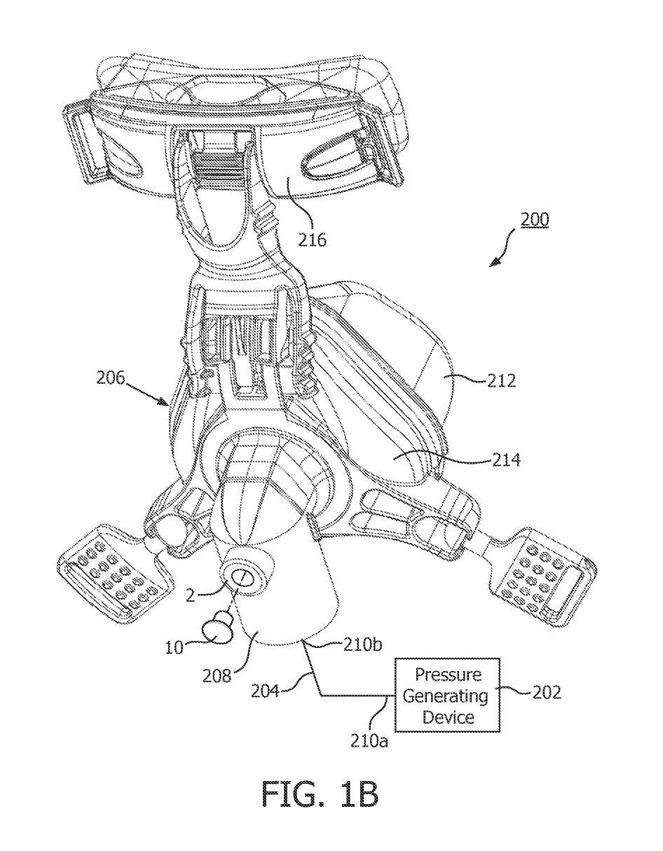

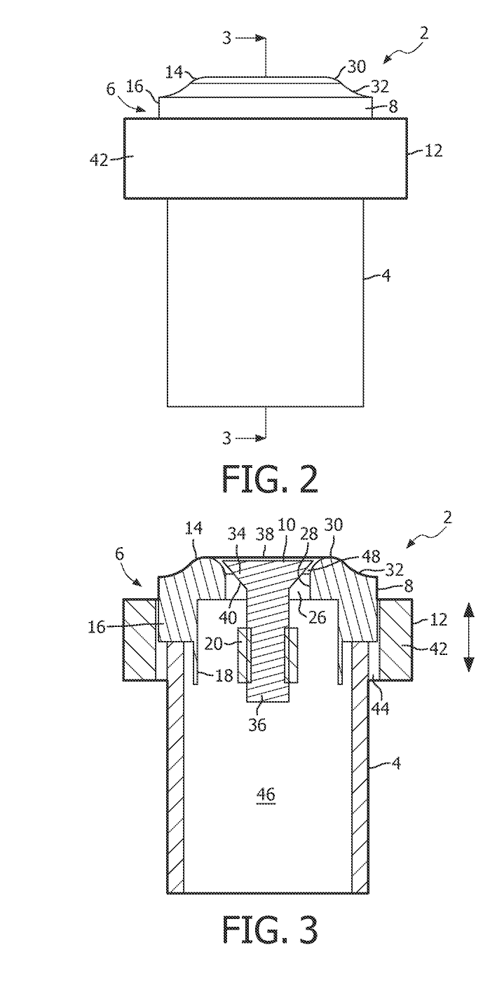

A system 200 adapted to provide a regimen of respiratory therapy to a patient according to one embodiment is generally shown in FIGS. 1A and 1B. System 200 includes pressure generating device 202, patient circuit 204, and patient interface device 206, and exhaust port assembly 208 having fluid flow control apparatus 2 described in detail elsewhere herein (FIGS. 2-18). Although system 200 is discussed as including pressure generating device 202, patient circuit 204, patient interface device 206, and exhaust port assembly 208 having fluid flow control apparatus 2 as shown, it is contemplated that other systems may be employed while remaining within the scope of the present invention. For example, and without limitation, a system in which the pressure generating device is coupled to a patient interface device having fluid flow control apparatus 2 integrated therein is contemplated.

Pressure generating device 202 is structured to generate a flow of breathing gas and may include, without limitation, ventilators, constant pressure support devices (such as a continuous positive airway pressure device, or CPAP device), variable pressure devices (e.g., BiPAp®, Bi-Flex®, or C-Flex™ devices manufactured and distributed by Respironics, Inc. of Murrysville, Pa.), and auto-titration pressure support devices.

Patient circuit 204 is structured to communicate the flow of breathing gas from pressure generating device 202 to patient interface device 206. Typically, patient circuit 204 includes a conduit or tube, a first end 210a of which couples with pressure generating device 202 and a second end 210b of which couples with patient interface device 206. In the current embodiment, second end 208b is coupled with the exhaust port assembly 208 which, in turn, is coupled with patient interface device 206.

Patient interface device 208 is typically a nasal or nasal/oral mask structured to be placed on and/or over the face of a patient. Any type of patient interface device 206, however, which facilitates the delivery of the flow of breathing gas to, and the removal of a flow of exhalation gas from, the airway of such a patient may be used while remaining within the scope of the present invention. In the embodiment shown in FIGS. 1A and 1B, patient interface device 206 includes cushion 212, rigid shell 214, and forehead support 216. Straps (not shown) may be attached to shell 214 and forehead support 216 to secure patient interface device 206 to the patient's head.

An opening in shell 214, to which exhaust port assembly 208 is coupled, allows the flow of breathing gas from pressure generator device 202 to be communicated to an interior space defined by shell 214 and cushion 212, and then, to the airway of a patient. The opening in shell 214 also allows the flow of exhalation gas (from the airway of such a patient) to be communicated to exhaust port assembly 208 in the current embodiment.

Although illustrated as a separate component in FIG. 1, it is contemplated that exhaust port assembly 10 may be incorporated into, for example and without limitation, patient interface 206 and/or patient circuit 204 while remaining within the scope of the present invention.

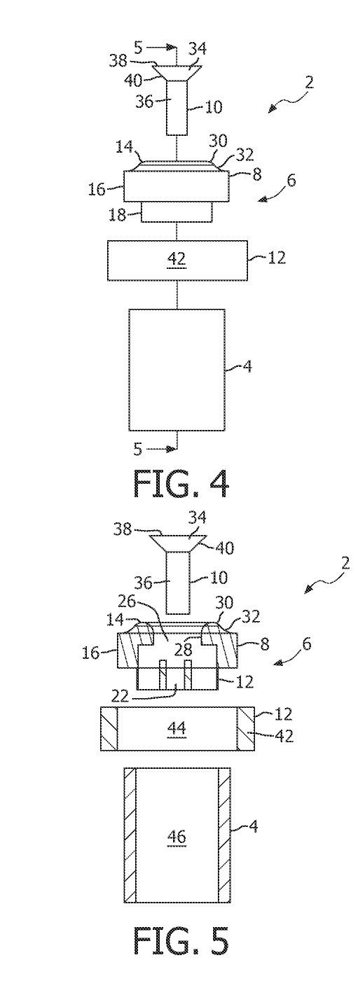

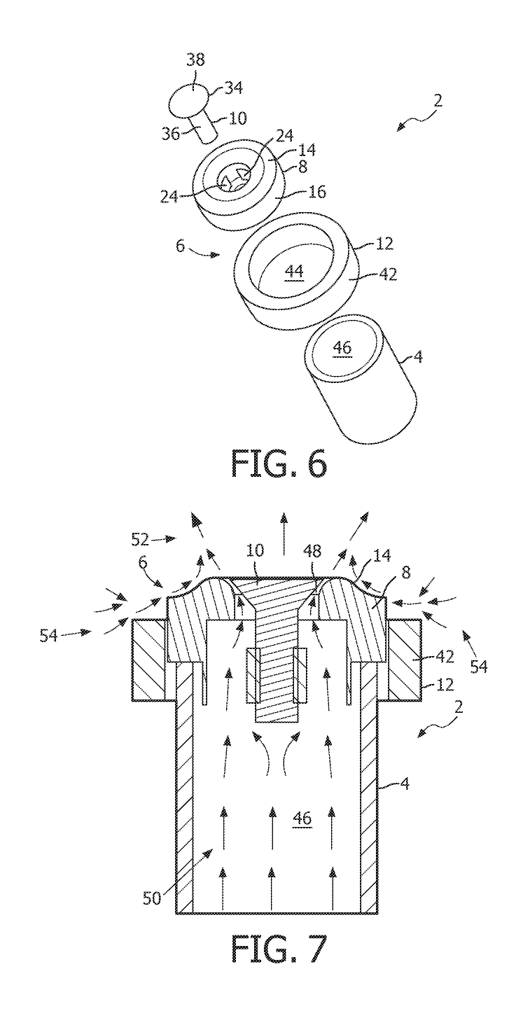

FIGS. 2 through 6 show various views of fluid flow control apparatus 2 according to one particular embodiment of the present invention. More specifically, FIG. 2 is a front elevational view, FIG. 3 is a front cross-sectional view (taken along lines 3-3 of FIG. 2), FIG. 4 is an exploded front elevational view, FIG. 5 is an exploded front cross-sectional view (taken along lines 5-5 of FIG. 5), and FIG. 6 is an exploded isometric view of fluid flow control apparatus 2. As described in greater detail herein, fluid flow control apparatus 2 enables a fluid, such as a liquid or a gas, to flow through it and enables a direction of the fluid flow adjacent to fluid flow control apparatus 2 to be selectively controlled.

Fluid flow control apparatus 2 includes main conduit 4 through which a pressured fluid is able to flow (from a source (not shown) of such a fluid), and nozzle assembly 6 coupled to main conduit 4. Nozzle assembly 6 includes external nozzle element 8, internal nozzle element 10 and switching element 12.

External nozzle element 8 has a generally annular shape and includes contoured top surface 14, intermediate wall portion 16, and bottom lip portion 18 having threaded internal wall 20 defining central bore 22 (FIG. 5). As seen in FIG. 6, holes 24 are provided in external nozzle element 8 to provide a path for fluid flow from main conduit 4. Contoured top surface 14 and inner wall portion 28 of intermediate wall portion 16 define main bore 26. As seen in FIG. 3, inner wall portion 28 extends parallel to a longitudinal axis of main bore 26. Contoured top surface 14 includes convex portion 30 connected to and adjacent to inner wall portion 28, and concave portion 32 connected to and adjacent to convex portion 30.

Internal nozzle element 10 includes conical (inverted) top portion 34 coupled to threaded, cylindrical bottom portion 36. Cylindrical bottom portion 36 extends downwardly from conical top portion 34 toward main conduit 4. Conical top portion 34 includes flat base 38 connected to external wall 40.

Switching element 12 is in the shape if an annular body including wall 42. Switching element 12 includes internal bore 44 defined by the interior of wall 42.

When assembled, bottom lip portion 18 of external nozzle element 8 is received within interior 46 of main conduit 4, and the bottom of intermediate wall portion 16 rests on the top edge of main conduit 4. In an exemplary embodiment, external nozzle element 8 is secured to main conduit 4 by a suitable means, such as a friction fit or an adhesive. Also, an airtight seal between external nozzle element 8 is secured to main conduit 4 is provided. Cylindrical bottom portion 36 of internal nozzle element 10 is received and held within central bore 22 of external nozzle element 8 as a result of mating between the threads of cylindrical bottom portion 36 and the threads of threaded internal wall 20. As a result of such threaded coupling, the position of internal nozzle element 10 relative to external nozzle element 8 is adjustable by rotating internal nozzle element 10.

According to an aspect of the present invention, internal nozzle element 10 is positioned so that a gap 48 exists between external wall 40 of conical top portion 34 and both inner wall portion 28 of intermediate wall portion 16 and convex portion 30 of contoured top surface 14. In addition, switching element 12 is positioned such that external nozzle element 8 and the top of main conduit 4 are received within internal bore 44 of switching element 12 as seen in FIG. 3. According to a further aspect of the present invention, when switching element 12 is positioned in this manner, it is movable relative to external nozzle element 8 and the top of main conduit 4 in the direction shown by the arrows in FIG. 3. This may be accomplished in a number of ways. In the embodiment shown, a friction fits exists between switching element 12 and external nozzle element 8 such that switching element 12 is held in place but may be manually moved relative to external nozzle element 8 and the top of main conduit 4 by application of a sufficient force in a direction parallel to the longitudinal axis of internal bore 44. Alternatively, switching element 12 may be moved automatically by way of, for example and without limitation, a pneumatic mechanism or electric motor acting upon switching element 12. Other arrangements for moving switching element 12 are also possible.

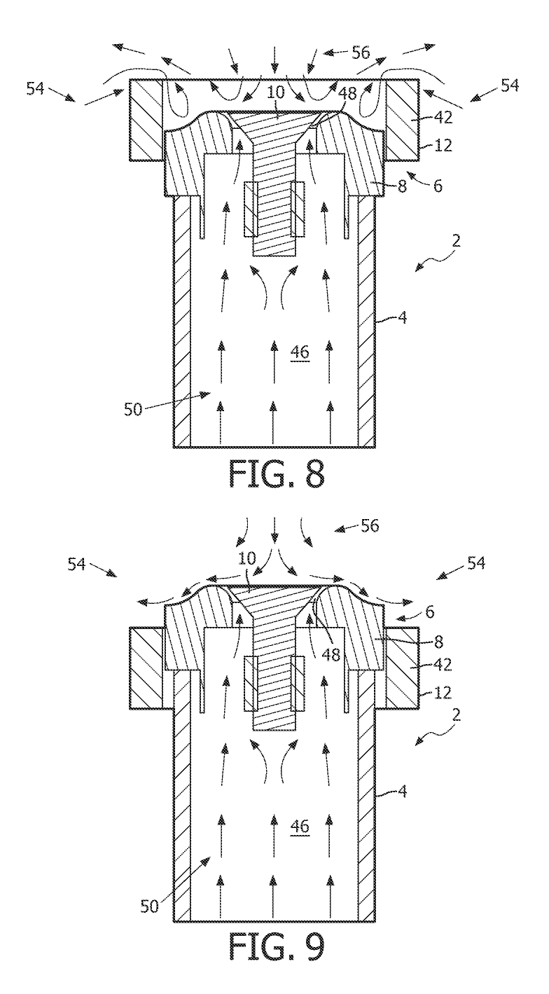

FIGS. 7, 8 and 9 illustrate operation of fluid flow control apparatus 2 according to one particular embodiment. Specifically, FIG. 7 shows fluid flow control apparatus 2 in an initial state, FIG. 8 shows fluid flow control apparatus 2 in a switching state, and FIG. 9 shows fluid flow control apparatus 2 in a switched state, all of which care described below.

Referring to FIG. 7, in the initial state, switching element 12 is positioned such that its top surface is located below contoured top surface 14 of external nozzle element 8. In the initial state, pressurized gas 50 is provided to main conduit 4. The flow of such pressurized gas 50 is shown by the arrows in FIG. 7. As seen in FIG. 7, pressurized gas 50 flows through gap 48 and forms 360° conical exhaust flow 52 flowing upward (forward) in a direction away from the top center of nozzle assembly 6. In addition, ambient gas (e.g., air) 54 surrounding nozzle assembly 6, also shown by arrows in FIG. 7, is drawn from the perimeter of nozzle assembly 6 and toward the top center of nozzle assembly 6 (referred to herein as a perimeter intake flow).

Referring to FIG. 8, in the intermediate, switching state, switching element 12 is moved (by, for example, one of the mechanisms described elsewhere herein) and positioned such that its top surface is located above contoured top surface 14 of external nozzle element 8. In such a position, at least part of wall 42 of switching element 12 blocks and disrupts the inward flow of ambient gas 54. In addition, as shown in FIG. 8, 360° conical exhaust flow 52 is no longer present. Instead, ambient gas (e.g., air) intake flow 56, shown by the arrows in FIG. 8, is present and flows in a downward (backward) direction toward the top center of nozzle assembly 6 that is generally parallel to the longitudinal axis of main conduit 4 (opposite the direction of 360° conical exhaust flow 52). Also, the direction of flow of ambient gas (e.g., air) 54 surrounding nozzle assembly 6 reverses and flows away from the top center of nozzle assembly 6 and toward the perimeter of nozzle assembly 6 (referred to herein as a perimeter outflow). This also results in an outward, perimeter exhaust flow of the pressurized gas 50.

Referring to FIG. 9, in the final, switched state, switching element 12 is moved (by, for example, one of the mechanisms described elsewhere herein) and returned to the position it was in the initial state. As shown in FIG. 9, the result is intake flow 56 in a direction downward (backward) and toward the top center of nozzle assembly 6, and perimeter outflow as described above which includes an exhaust flow of pressurized gas 50 through gap 48.

Thus, in progressing from the initial state to the switched state, the flow of gas or gases surrounding nozzle assembly goes from perimeter intake and exhaust flow upward (forward) and away from the top center of nozzle assembly 6 to flow downward (backward) and toward the top center of nozzle assembly 6 and perimeter outflow including exhaust. The various flows and changes therein just described are believed to occur due to the Coanda effect, which is a principle that states that a moving stream of fluid in contact with a curved surface will tend to follow the curvature of the surface rather than to travel in a straight line.

To return to the initial state (reset), either the flow of pressurized gas 50 can be turned off and back on again, or the top of nozzle assembly 6 can be completely blocked for a moment. Moving switching element 12 (as described above) upward in the switched state will not cause a return to the initial state (reset) to occur.

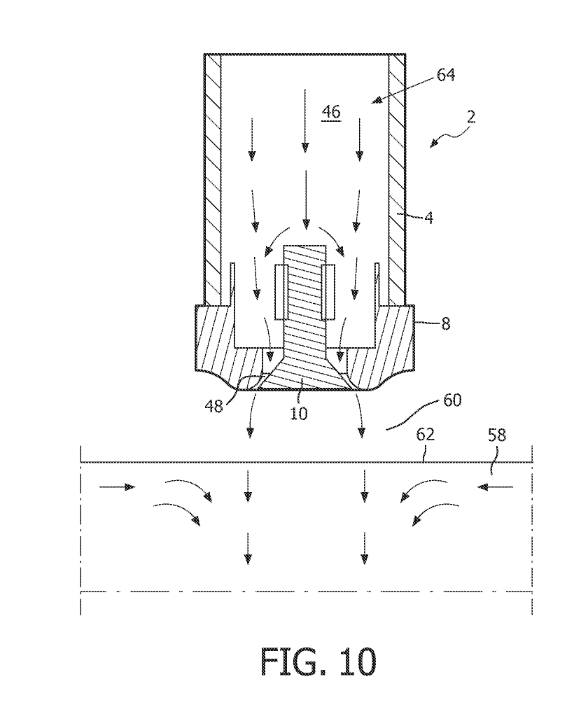

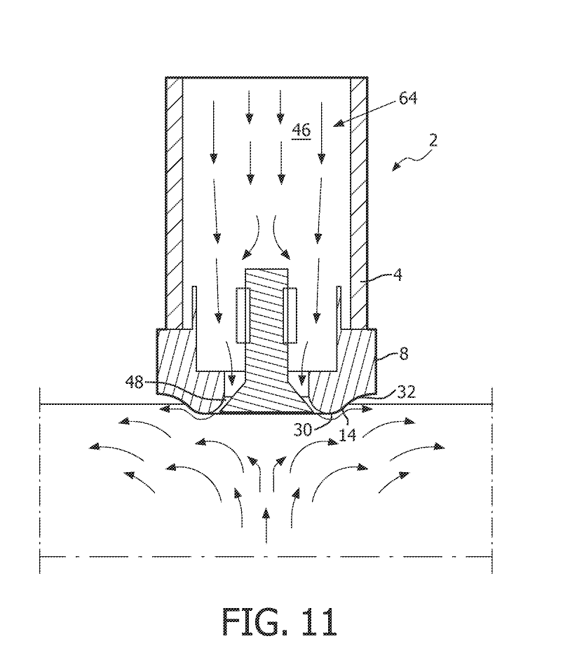

FIGS. 10 and 11 illustrate operation of a slightly modified fluid flow control apparatus 2 according to another embodiment. In this embodiment, fluid flow control apparatus 2 is employed without using switching element 12 (in the implementation shown, switching element 12 is absent; it is also possible in this embodiment to provide switching element 12 and just not use it as described above). In addition, the pressurized fluid involved in this embodiment is a liquid.

FIG. 10 shows an initial state in this embodiment wherein modified fluid flow control apparatus 2 is positioned above a body of liquid 58. As seen in FIG. 10, in this state, there is an air gap 60 between top surface 62 of body of liquid 58 and the top of external nozzle element 8. Also, pressurized liquid 64 flows through main conduit 4 and through gap 48 as shown by the arrows in FIG. 10. This results in a fluid flow in body of liquid 58 (shown by the arrows in FIG. 10) that goes from outside the perimeter of external nozzle element 8 and fluid flow control apparatus 2 toward inside the perimeter of external nozzle element 8 and fluid flow control apparatus 2 and downward and away from fluid flow control apparatus 2.

FIG. 11 shows a switched state in this embodiment wherein at least a portion of external nozzle element 8 of modified fluid flow control apparatus 2 is inserted into body of liquid 58 and air gap 60 between top surface 62 of body of liquid 58 and the top of external nozzle element 8 has been eliminated. In one embodiment, all of convex portion 30 and all or part of concave portion 32 of contoured top surface 14 lie beneath top surface 62 of body of liquid 58. As seen in FIG. 11, in this state, the fluid flow in body of liquid 58 (shown by the arrows in FIG. 11) reverses and now goes upward and toward fluid flow control apparatus 2 and from inside the perimeter of external nozzle element 8 and fluid flow control apparatus 2 toward outside the perimeter of external nozzle element 8 and fluid flow control apparatus 2. If fluid flow control apparatus 2 is raised to reintroduce air gap 60, the initial state described above is restored. Again, the various flows and changes therein just described are believed to occur due to the Coanda effect.

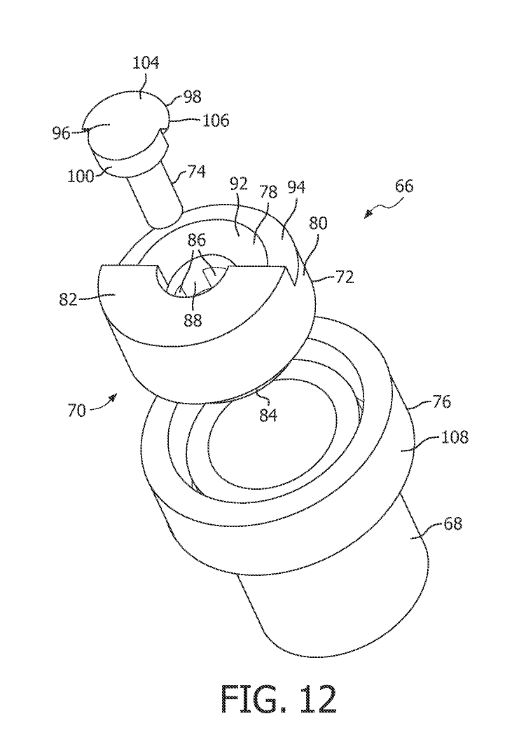

FIG. 12 is an exploded view of fluid flow control apparatus 66 according to an alternative embodiment of the present invention. Fluid flow control apparatus 66 includes main conduit 68 through which a pressured fluid is able to flow (from a source (not shown) of such a fluid), and nozzle assembly 70 coupled to main conduit 68. Nozzle assembly 70 includes external nozzle element 72, internal nozzle element 74 and switching element 76.

External nozzle element 72 has a generally annular shape and includes contoured semicircular top surface 78, intermediate wall portion 80, flat top surface 82 and bottom lip portion 84. As seen in FIG. 12, holes 86 are provided in external nozzle element 72 to provide a path for fluid flow from main conduit 68. Contoured semicircular top surface 78 and inner wall portion 88 of intermediate wall portion 80 define a main bore. Contoured semicircular top surface 78 includes convex portion 92 connected to and adjacent to inner wall portion 88, and concave portion 94 connected to and adjacent to convex portion 92.

Internal nozzle element 74 includes top portion 96 having first side 98 having a partial conical (inverted) shape and second side 100 having a cylindrical shape. Top portion 96 is coupled to cylindrical bottom portion 102. Cylindrical bottom portion 102 extends downwardly from top portion 96 toward main conduit 68. First side 98 includes flat base portion 104 connected to external wall 106.

Switching element 76 is in the shape if an annular body including wall 108. Switching element 76 includes an internal bore defined by the interior of wall 108.

When assembled, internal nozzle element 74 is positioned so that a gap exists between external wall 106 and both inner wall portion 88 of intermediate wall portion 80 and convex portion 92 of contoured semicircular top surface 78. Operation of fluid flow control apparatus 66 is similar to operation of fluid flow control apparatus 2 as described elsewhere herein. Fluid flow control apparatus 66 may be used without switching element 76 in liquid applications as described elsewhere herein (FIGS. 10 and 11).

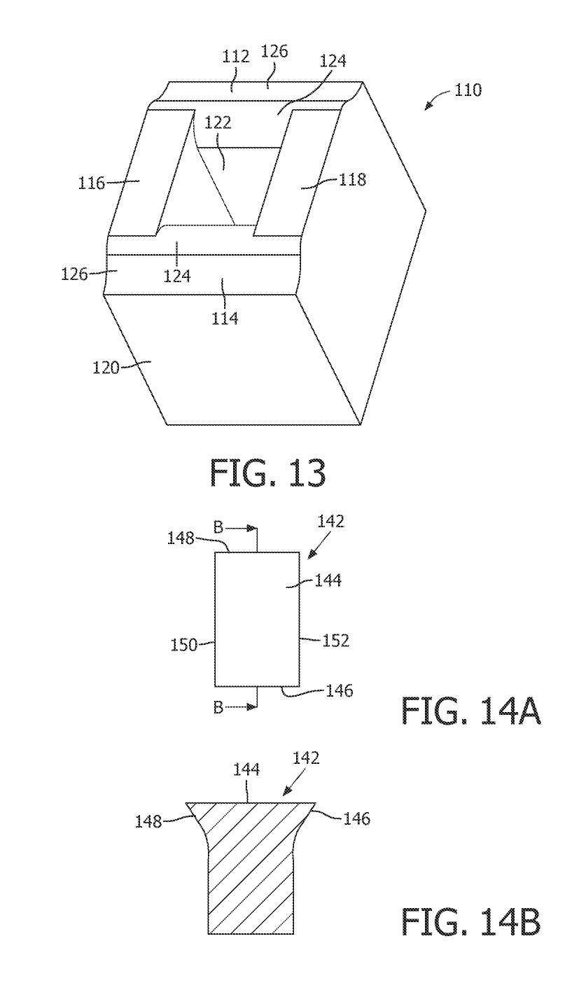

FIG. 13 is an isometric view of external nozzle element 110 according to an alternative embodiment that may be used with an alternative fluid flow control apparatus having the same principal of operation as fluid flow control apparatus 2. External nozzle element 110 has a generally rectangular shape and includes contoured top side surfaces 112 and 114 located opposite one another, flat top side surfaces 116 and 118 located opposite one another, and intermediate wall portion 120. Contoured top side surfaces 112 and 114, flat top side surfaces 116 and 118 and inner wall portion 122 of intermediate wall portion 120 define a main bore. Contoured top side surfaces 112 and 114 each includes convex portion 124 connected to and adjacent to inner wall portion 122, and concave portion 126 connected to and adjacent to convex portion 124. External nozzle element 110 is structured to be employed in an alternative fluid flow control apparatus that includes internal nozzle element 142 shown in FIGS. 14A and 14B and a switching element (not shown) that each has a shape that is complementary to the shape of external nozzle element 110. As seen in the top plan view of internal nozzle element 142 shown in FIG. 14A and in the cross-sectional view of internal nozzle element 142 shown in FIG. 14B (taken along lines B-B in FIG. 14A), internal nozzle element 142 includes top portion 144 having opposing inwardly angled sides 146, 148 and opposing flat sides 150, 152. A gap will be formed between opposing inwardly sides 146, 148 and inner wall portions 122 and convex portions 124. Operation of such a fluid flow control apparatus is similar to operation of fluid flow control apparatus 2 as described elsewhere herein.

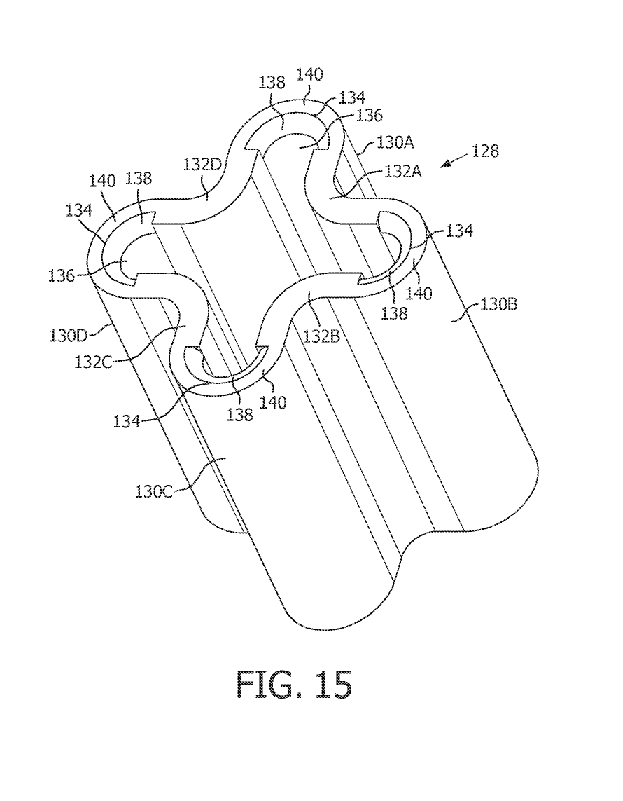

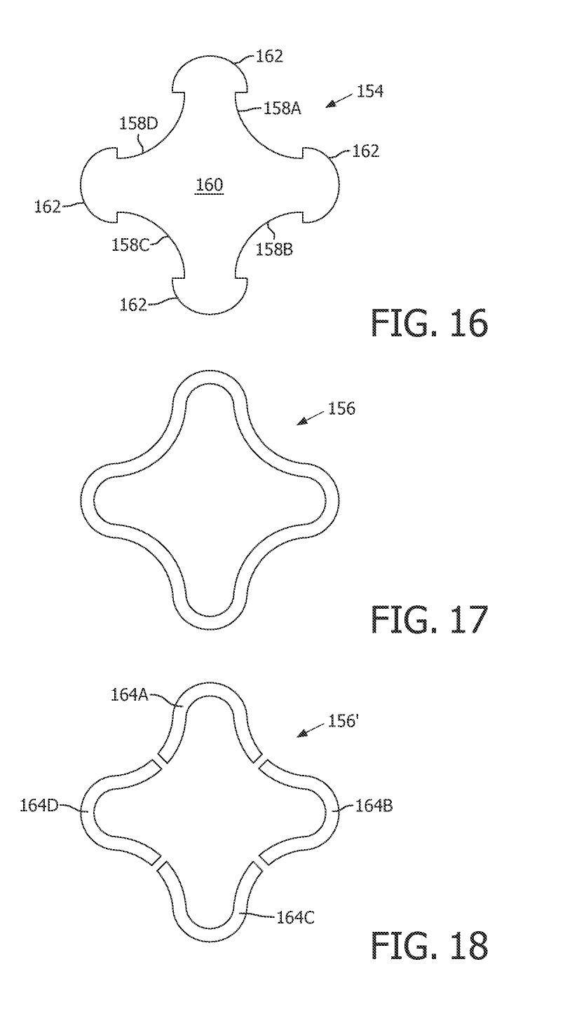

FIG. 15 is an isometric view of external nozzle element 128 according to a further alternative embodiment that may be used with a further alternative fluid flow control apparatus having the same principal of operation as fluid flow control apparatus 2. External nozzle element 128 has a “flower petal” shape and includes four semi-cylindrical sections 130A, 130B, 130C and 130D coupled to one another by curved joining sections 132A, 132B, 132C and 132D as shown. Each semi-cylindrical section 130A, 130B, 130C, 130D includes contoured semicircular top surface 134 and inner wall portion 136. Contoured top side surfaces 112 and 114, flat top side surfaces 116 and 118 and inner wall portion 122 of intermediate wall portion 120 define a main bore. Contoured semicircular top surfaces 134, inner wall portions 136 and curved joining sections 132A, 132B, 132C and 132D define a main bore. Contoured semicircular top surfaces 134 each include convex portion 138 connected to and adjacent to inner wall portion 136, and concave portion 140 connected to and adjacent to convex portion 138. External nozzle element 128 is structured to be employed in an alternative fluid flow control apparatus that includes internal nozzle element 154 shown in FIG. 16 and switching element 156 shown in FIG. 17 (top plan view) that each has a shape that is complementary to the shape of external nozzle element 128. As seen in FIG. 16, which is a top plan view of internal nozzle element 154, top portion 160 of internal nozzle element 154 includes four sections 158A, 158B, 158C, and 158D, each having semicircular side sections 162 having a partial conical (inverted) shape. A gap will be formed between each of the semicircular side sections 162 and the corresponding inner wall portion 136 and convex portion 138. Operation of such a fluid flow control apparatus is similar to operation of fluid flow control apparatus 2 as described elsewhere herein.

In an alternative embodiment, switching element 156′ may be provided that includes four separate and independently moveable sections 164A, 164B, 164C, and 164D. With such at configuration, each semi-cylindrical section 130A, 130B, 130C, 130D can be selectively and independently switched as described elsewhere herein by moving the corresponding section 164A, 164B, 164C, 164D of switching element 156′.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

In addition, while fluid flow control apparatus 2 has be shown in FIGS. 1A and 1B as being employed in system 200 having patient interface device3 206, other applications of fluid flow control apparatus 2 are contemplated and are within the scope of the present invention. For instance, and without limitation, fluid flow control apparatus 2 and the methods described herein may be used in any of the following gaseous applications: ventilation (e.g., building, enclosure), gaseous mixing (e.g., oxygen enrichment, industrial processes), gas removal (e.g., fume extraction, exhaust removal), gas diffusion (e.g., jet diffuser, exhaust diffuser), special effects (e.g., fog machine, magician's tricks), gas propulsion (e.g., jet engine, compressed air engine), and gas flow reversal (e.g., pipeline, jet exhaust). In addition, and without limitation, fluid flow control apparatus 2 and the methods described herein may be used in any of the following liquid applications: liquid mixing (e.g., paint, industrial processing, chemical), liquid refinement/separation (e.g., crude oil, waste processing), liquid aeration (e.g., aquarium, waste processing), liquid purification (desalination, water purification), liquid distillation (e.g., alcohol, industrial processes), liquid cooling (e.g., industrial processing, energy generation), liquid propulsion (e.g., jet-ski motor, scuba equipment), liquid dispersion (e.g., humidification, aerosol generator), and vapor deposition (e.g., silicon chip manufacture, diamond coating).

What is claimed is:

1. A patient interface device comprising:
(a) a cushion;
(b) a mask frame supporting the cushion;
(c) a patient circuit coupled to the mask frame adapted to carry a flow of gas; and
(d) a fluid flow control apparatus coupled to the mask frame or the patient circuit, comprising:
(1) a main conduit structured to receive a pressurized fluid; and
(2) a nozzle assembly coupled to the main conduit, the nozzle assembly comprising:
(i) an external nozzle element having a contoured top surface and a main bore in fluid communication with an interior of the main conduit,
(ii) an internal nozzle element received within the main bore, wherein a gap is provided between the internal nozzle element and the external nozzle element, and
(iii) a switching element selectively moveable between a first position and a second position, wherein in the first position, the switching element is positioned below the contoured top surface of the external nozzle element, and wherein in the second position, at least a portion of the switching element is positioned adjacent to or above the contoured top surface of the external nozzle element.

2. The patient interface device according to claim 1, wherein in the second position, the at least a portion of the switching element is positioned above the contoured top surface of the external nozzle element.

3. The patient interface device according to claim 1, wherein the external nozzle element has an annular shape, and wherein the switching element comprises an annular body.

4. The patient interface device according to claim 3, wherein the contoured top surface is semicircular and extends along half of an entire top surface of the external nozzle element.

5. The patient interface device according to claim 1, wherein the contoured top surface comprises a convex portion adjacent to an inner wall portion of the external nozzle element, and a concave portion adjacent to the convex portion.

6. The patient interface device according to claim 5, wherein the inner wall portion extends parallel to a longitudinal axis of the main bore.

7. The patient interface device according to claim 5, wherein the internal nozzle element comprises a conical top portion having a flat base connected to an external wall, the external wall extending downwardly from the flat base toward the main conduit, wherein the gap is between the external wall and the convex portion and the inner wall portion of the external nozzle element.

8. The patient interface device according to claim 7, wherein the external nozzle element has an annular shape and wherein the switching element comprises an annular body and wherein the conical top portion is coupled to a cylindrical bottom portion, and wherein the cylindrical bottom portion is received and held within an internal wall of the external nozzle element.

9. The patient interface device according to claim 8, wherein both the cylindrical bottom portion and the internal wall are threaded.

10. The patient interface device according to claim 1, wherein a position of the internal nozzle element relative to the external nozzle element is selectively adjustable.

11. The patient interface device according to claim 1, wherein the external nozzle element has a rectangular shape, wherein the contoured top surface comprises a first contoured surface portion on a first side of the external nozzle element and a second contoured surface portion on a second side of the external nozzle element opposite the first side.

12. The patient interface device according to claim 1, wherein the external nozzle element includes a plurality of semi-cylindrical sections coupled to one another by a plurality of curved joining sections, wherein the contoured top surface comprises a plurality of contoured surface portions, each contoured surface portion being provided on a top of a respective one of the semi-cylindrical sections.

13. A method of controlling fluid flow, comprising:
providing a fluid flow control apparatus having a main conduit and a nozzle assembly coupled to the main conduit, the nozzle assembly including an external nozzle element having a contoured top surface and a main bore in fluid communication with an interior of the main conduit and an internal nozzle element received within the main bore, wherein a gap is provided between the internal nozzle element and the external nozzle element;
providing a pressurized gas within the main conduit in an initial state, wherein in the initial state: (i) an exhaust flow flows forward and out of the nozzle assembly in a direction away from a top center of the nozzle assembly, and (ii) ambient gas surrounding the nozzle assembly is drawn from a perimeter of the nozzle assembly and toward the top center of the nozzle assembly; and
causing the fluid flow control apparatus to move to a switched state by disrupting a flow of the ambient gas toward the top center of the nozzle assembly, wherein in the switched state: (i) an ambient gas intake flow is present and flows in a backward direction toward the top center of nozzle assembly, (ii) ambient gas surrounding the nozzle assembly flows away from the top center of nozzle assembly and toward the perimeter of the nozzle assembly, and a second exhaust flow flows out of the nozzle assembly and toward the perimeter of the nozzle assembly.

14. The method according to claim 13, wherein the contoured top surface comprises an a convex portion adjacent to an inner wall portion of the external nozzle element, and a concave portion adjacent to the convex portion, and wherein the internal nozzle element comprises a conical top portion having a flat base connected to an external wall, the external wall extending downwardly from the flat base toward the main conduit, wherein the gap is between the external wall and the convex portion and the inner wall portion of the external nozzle element.

15. A patient interface device comprising:
(a) a cushion;
(b) a mask frame supporting the cushion;
(c) a patient circuit coupled to the mask frame adapted to carry a flow of gas; and
(d) a fluid flow control apparatus coupled to the mask frame or the patient circuit, comprising:
(1) a main conduit structured to receive a pressurized fluid; and
(2) a nozzle assembly coupled to the main conduit, the nozzle assembly comprising:
(i) an external nozzle element having a contoured top surface and a main bore in fluid communication with an interior of the main conduit, wherein the contoured top surface comprises a convex portion adjacent to an inner wall portion of the external nozzle element, and a concave portion adjacent to the convex portion, and
(ii) an internal nozzle element received within the main bore, wherein a gap is provided between the internal nozzle element and the external nozzle element.

16. The patient interface device according to claim 15, wherein the external nozzle element has an annular shape.

17. The patient interface device according to claim 15, wherein the inner wall portion extends parallel to a longitudinal axis of the main bore.

18. The patient interface device according to claim 16, wherein the internal nozzle element comprises a conical top portion having a flat base connected to an external wall, the external wall extending downwardly from the flat base toward the main conduit, wherein the gap is between the external wall and the convex portion and the inner wall portion of the external nozzle element.

19. The patient interface device according to claim 18, wherein the conical top portion is coupled to a cylindrical bottom portion, and wherein the cylindrical bottom portion is received and held within an internal wall of the external nozzle element.

20. The patient interface device according to claim 19, wherein both the cylindrical bottom portion and the internal wall are threaded.

21. The patient interface device according to claim 15, wherein a position of the internal nozzle element relative to the external nozzle element is selectively adjustable.

22. The patient interface device according to claim 15, wherein the contoured top surface is semicircular and extends along half of an entire top surface of the external nozzle element.

23. The patient interface device according to claim 15, wherein the external nozzle element has a rectangular shape, wherein the contoured top surface comprises a first contoured surface portion on a first side of the external nozzle element and a second contoured surface portion on a second side of the external nozzle element opposite the first side.

24. The patient interface device according to claim 15, wherein the external nozzle element includes a plurality of semi-cylindrical sections coupled to one another by a plurality of curved joining sections, wherein the contoured top surface comprises a plurality of contoured surface portions, each contoured surface portion being provided on a top of a respective one of the semi-cylindrical sections.

25. A method of controlling fluid flow, comprising:
providing a fluid flow control apparatus having a main conduit and a nozzle assembly coupled to the main conduit, the nozzle assembly including an external nozzle element having a contoured top surface and a main bore in fluid communication with an interior of the main conduit and an internal nozzle element received within the main bore, wherein a gap is provided between the internal nozzle element and the external nozzle element;
providing a pressurized liquid within the main conduit in an initial state, wherein in the initial state: (i) the pressured liquid flows though the gap and out of the nozzle assembly, (ii) the nozzle assembly is positioned above a top surface of a body of liquid, (iii) a fluid flow in the body of liquid goes from outside a perimeter of the external nozzle element to inside the perimeter of the external nozzle element, and downward and away from the fluid flow control apparatus; and
causing the fluid flow control apparatus to move to a switched state by introducing the nozzle assembly into the body of liquid, wherein in the switched state: (i) the pressured liquid flows though the gap and out of the nozzle assembly, (ii) the fluid flow in the body of liquid reverses and goes upward and toward the fluid flow control apparatus and from inside the perimeter of the external nozzle element to outside the perimeter of the external nozzle element.

26. The method according to claim 25, wherein the contoured top surface comprises a convex portion adjacent to an inner wall portion of the external nozzle element, and a concave portion adjacent to the convex portion, and wherein the internal nozzle element comprises a conical top portion having a flat base connected to an external wall, the external wall extending downwardly from the flat base toward the main conduit, wherein the gap is between the external wall and the convex portion and the inner wall portion of the external nozzle element.

27. The method according to claim 25, wherein in the switched state all of the convex portion and all or part of the concave portion of the contoured top surface lie beneath a top surface of the body of liquid.
